(19) United States
(12) Patent Application Publication
(10) Pub. No.: US 2019/0098896 A1
(43) Pub. Date: Apr. 4, 2019

(54) CONCENTRATE COMPRISING A MEL, AN ALKYL POLYGLUCOSIDE AND MONOPROPYLENE GLYCOL

(71) Applicant: OLEON NV, Evergem (Ertvelde) (BE)

(72) Inventors: Pierre RAVIER, COMPIEGNE (FR); Sophie DEPREY, MARGNY-LES-COMPIEGNE (FR)

(21) Appl. No.: 16/085,367

(22) PCT Filed: Mar. 17, 2017
(86) PCT No.: PCT/EP2017/056463

§ 371 Date: Sep. 14, 2018

(30) Foreign Application Priority Data
Mar. 17, 2016 (FR) 1652287

(51) Int. Cl. A01N 25/30; A01N 25/04; A01N 25/10; A01N 43/16; A61K 8/49; A61K 8/73

(52) U.S. Cl. CPC A01N 25/30; A01N 25/04; A61K 8/73; A01N 43/16; A61K 8/4973; A01N 25/10

(57) ABSTRACT

The present invention relates to a concentrate comprising at least one lipid of mannosylerythritol (MEL), at least one alkypolyglucoside and monopropylene glycol, to the method for preparing same and to the uses thereof, in particular as an adjuvant.

The present invention relates to a concentrate, to a composition comprising it, and to a solution comprising it. The present invention also relates to a process for the preparation of the concentrate, of the composition, and of the solution according to the invention, and uses thereof, in particular use of the concentrate according to the invention as adjuvant.

Adjuvants are generally defined as substances having the role of improving the physicochemical properties of solutions and more particularly of aqueous solutions comprising them. In the present application, by “solution” is meant more particularly an aqueous solution, which may or may not be homogeneous (for example, an emulsion, a dispersion or a suspension). By way of examples of adjuvants, penetrating agents, moistening agents, adhesive agents, anti-rebound agents, dispersants, anti-leaching agents, or also wetting agents may be mentioned. Wetting agents have the property of promoting the spreading of the solutions comprising them, and they therefore find an application in many fields. The use of wetting agents is in particular known in the cleaning industry, for example in solutions for household maintenance or cleaning, or also in the cosmetics industry. The use of wetting agents is also of particular interest in the industry for the protection of crops (agricultural) and non-agricultural areas such as parks, gardens, etc. In fact, the use of wetting agents in phytosanitary solutions, for example herbicide, fungicide, or insecticide solutions or also solutions stimulating the defenses of plants, improves the spreading of these solutions. This results, on the one hand, in a reduction in the quantity of phytosanitary solution to be sprayed on crops, and on the other hand in an improvement in the effectiveness of the phytosanitary solutions. The use of wetting agents thus allows a reduction in the efforts and costs associated with the treatment of crops.

Moreover, whatever the types of industry in which they are used, it is preferable that the wetting agents are environmentally friendly compounds and are less toxic for the operators using them. This is particularly important when they are present in phytosanitary emulsions, as these are generally spread over crops in significant quantities, so that it is preferable or even necessary, that the wetting agents are ecologically advantageous, and in particular biodegradable.

Currently, there is still a need for concentrates with a particularly high capacity for increasing the wetting power of solutions, such as phytosanitary solutions, that are in addition environmentally friendly.

The work of the inventor has made it possible to demonstrate that a specific concentrate had the effect of greatly increasing the wetting power of a solution when it was added to the latter, so that its use as a wetting agent, for example in phytosanitary solutions, would be particularly advantageous. Moreover, this concentrate comprises biodegradable components, and therefore has a good environmental profile.

The invention therefore relates to a concentrate comprising:
at least one mannosylerythritol lipid (MEL);
at least one alkyl polyglucoside, and
monopropylene glycol.

According to the invention, a “mannosylerythritol lipid” (also called “MEL”) is a surfactant belonging to the class of glycolipids. More particularly, a MEL is an amphiphilic molecule the hydrophilic part of which is formed by a mannosylerythritol residue, and the hydrophobic part of which is formed by at least one fatty acid.

More particularly, by “MEL”, is meant a molecule having the following general formula (I):

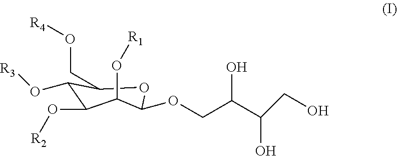

in which:
R₁ and R₂, identical or different, represent an unsaturated or saturated fatty acid, and
R₃ and R₄, identical or different, represent an acetyl group or a hydrogen atom.

Preferably, in the present invention, by “MEL”, is meant a molecule of the following formula (II):

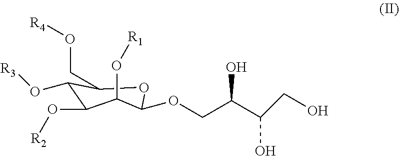

in which:
R₁ and R₂, identical or different, represent an unsaturated or saturated fatty acid, and
R₃ and R₄, identical or different, represent an acetyl group or a hydrogen atom.

The formulae (I) and (II) above can represent several molecules, each molecule therefore being a MEL. By “MELs”, is meant at least two different molecules of formulae (I), and more particularly of formula (II).

The MELs are generally classified in four classes of molecules, denoted A to D, according to their degree of acetylation in positions R₃ and R₄. The class of the MELs-A comprises the molecules of formulae (I) or (II) having two acetyl groups in positions R₃ and R₄. The class of the MELs-B and the class of the MELs-C comprise the molecules of formulae (I) or (II) having a single acetyl group in positions R₄ and R₃ respectively. Finally, the class of the MELs-D comprises the molecules of formulae (I) or (II) not having an acetyl group (R₃═R₄═H).

As well as by their degree of acetylation, the MELs can vary in their structure, by the nature of the fatty acids which comprise their hydrophobic part. This variation is generally a function of the process implemented for obtaining the MELs.

The MELs are generally obtained by processes implementing the culture of fungi, and more particularly of yeasts.

Advantageously, the MELs to which the present application relates are obtained by a fermentation process, comprising the following steps:
culturing a fungal strain and more particularly a yeast strain in the presence of a carbon source in order to obtain MELs; and
recovering the MELs thus obtained.

The strains from which it is possible to obtain MELs are well known to a person skilled in the art. By way of example, it is known to use fungal strains of the genus Pseudozyma or of the genus Ustilago, in order to obtain MELs.

Advantageously, the strains used in the fermentation process described above, making it possible to obtain MELs, are fungal strains belonging to the genus Pseudozyma. Preferably, the strain is Pzeudozyma antartica or Pzeudozyma aphidis.

Such strains are usually cultured in a reactor in a medium comprising glucose, water and/or salts (such as magnesium sulphate, monopotassium phosphate and/or ammonium nitrate).

Advantageously, the different components of the medium (glucose and strains in particular) are sterilized separately before introduction into the reactor.

The temperature of the medium is preferably comprised between 20 and 35° C., more preferentially between 25 and 30° C.

Advantageously, the carbon source allowing the production of MELs by the strain is an oil, such as a vegetable oil. Preferably, the source of carbon is a soya oil or even more preferentially a rapeseed oil. These oils are particularly rich in fatty acids comprising a carbon-containing chain with 18 carbon atoms, such as oleic, linoleic and/or linolenic acid, as well as, to a lesser degree, in fatty acids comprising a carbon-containing chain with 16 carbon atoms, such as in palmitic acid.

The recovery of the MELs following the culture step can comprise a step of separating the MELs from the other components of the medium. This step can be done by standard separation methods known to a person skilled in the art.

Advantageously, the recovery of the MELs can comprise one or more of the following separation methods:
settling;
evaporation of water or drying;
filtration; and/or
centrifugation.

The concentrate according to the invention also comprises at least one alkyl polyglucoside.

The alkyl polyglucosides are generally used as surfactants. They have the advantage that they can be prepared from renewable materials, they are non-toxic and they are easily biodegradable.

The concentrate according to the invention also comprises monopropylene glycol.

Monopropylene glycol (also called 1,2-dihydroxypropane) is a solvent used conventionally in various industries: food, pharmaceutical, air conditioning, aeronautical.

By “solvent”, is meant a liquid under normal temperature and pressure (NTP) conditions, which has the property of dissolving, diluting or extracting other substances without causing a chemical change of these substances and without itself undergoing any change.

When it is added to a solution, the concentrate according to the invention makes it possible to improve in particular the wetting power of this solution. More particularly, it was demonstrated by the inventors that, surprisingly, a solution comprising the concentrate according to the invention, i.e. comprising a combination of at least one MEL, at least one alkyl polyglucoside and monopropylene glycol, had excellent wetting power. This wetting power of the solution comprising the concentrate according to the invention is in particular greater than that of a solution comprising one, or a combination of two of these three components. Thus, when a solution comprising the concentrate according to the invention is applied onto a solid surface (such as a hydrophobic flat surface), the wetting of this solid surface by this solution is greater than the wetting of the same surface by a solution only comprising one, or a combination of two of the three components of the concentrate according to the invention.

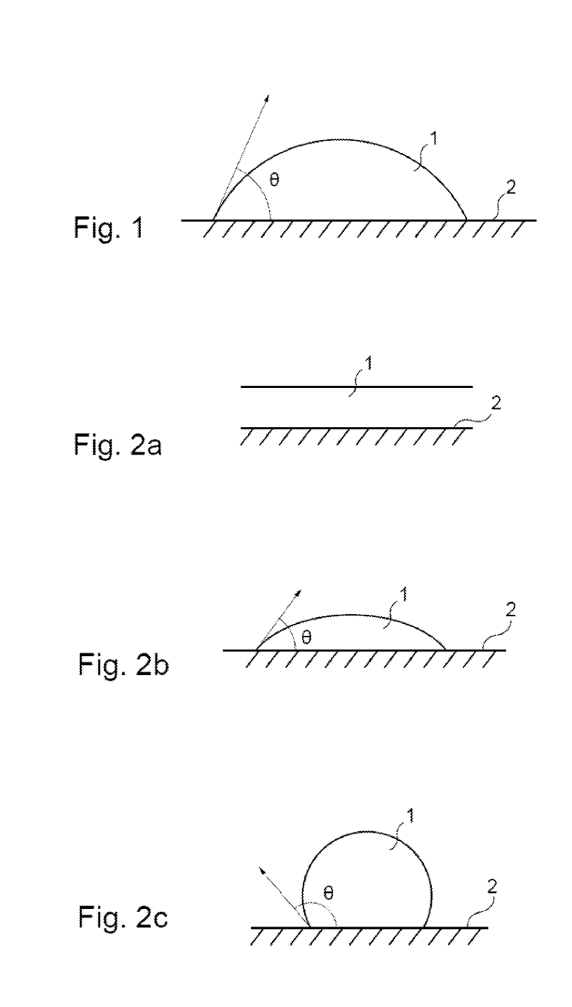

In the context of the present application:
by “wetting”, is meant the spreading ability of a liquid over a solid;
by “surface tension” of a liquid, is meant the force exerted at the interface between this liquid and a solid;
by contact angle of a drop of liquid 1 deposited on a flat solid surface 2, is meant the angle Θ formed by the tangent to the drop of liquid 1 at the point of contact with the flat solid surface 2, as shown in FIG. 1.

By way of example, when a liquid, such as a drop of solution, and a solid, such as a plant wall or leaf, are placed in contact, the ability of the liquid to wet the solid, i.e. to spread or become distributed over it, will depend directly on the force exerted at the interface between liquid and solid, which is generally defined as the surface tension. The surface tension therefore represents the force making it possible for the liquid to adhere to the solid, or prevent it becoming distributed over it. Thus the higher surface tension, the less the liquid is capable of wetting the solid in question.

Several cases in point can therefore be shown in order to illustrate the idea of wetting. FIGS. 2a to 2c represent more particularly three cases in point.

Thus, as shown in FIG. 2a, when a drop of liquid 1 falls onto a flat solid surface 2, it can achieve a total wetting of this surface 2, i.e. become distributed over the entire surface thereof, by forming a film with a contact angle Θ equal to 0 with said surface 2. Alternatively, the drop 1 can partially wet the surface 2 (FIG. 2b), i.e. it does not become totally distributed over the latter, by forming a drop with a contact angle Θ comprised between 0 and 90° with said surface 2. Finally, the drop 1 may not wet the surface 2 at all (FIG. 2c), i.e. it does not become distributed over it, by forming a drop with a contact angle Θ greater than 90° with said surface 2.

In particular, when monopropylene glycol is added to a solution comprising an alkyl polyglucoside, or to a solution comprising a MEL, this does lead to an increase in the wetting power of these solutions. Now, surprisingly, the inventors have found that when monopropylene glycol is added to a solution comprising an alkyl polyglucoside and a MEL, so that the solution comprises the concentrate according to the invention, the wetting power of said solution is increased considerably.

The effect of the concentrate according to the invention on the wetting power of a solution is described more fully in Example 2.

Advantageously, the alkyl polyglucoside present in the concentrate according to the invention is a molecule of the following formula (III):

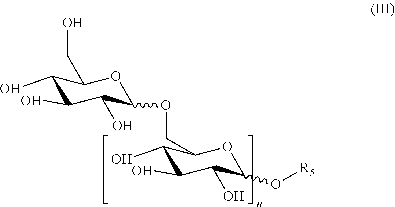

in which:
n is an integer comprised between 0 and 5,
R₅ is an alkyl chain comprising between 5 and 20 carbon atoms.

It will be noted that in the context of the present application, and unless stated otherwise, the value ranges indicated are inclusive.

Preferably, the alkyl polyglucoside included in the concentrate according to the invention is a molecule of formula (III) in which:
n is an integer comprised between 0 and 5,
R₅ is an alkyl chain comprising between 8 and 14 carbon atoms.

Preferably, the at least one alkyl polyglucoside present in the concentrate according to the invention is a molecule of formula (III), in which n is equal to 0 and R₅ is an alkyl chain comprising 11 carbon atoms (also called undecyl group). An alkyl polyglucoside of this kind has the following formula (IV):

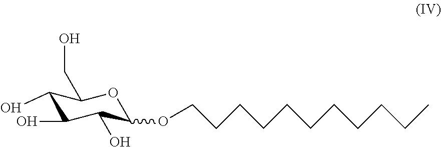

Advantageously, the total quantity of alkyl polyglucoside(s) in the concentrate according to the invention is comprised between 0.1 and 25% by weight with respect to the total weight of the concentrate.

By “total quantity of alkyl polyglucoside(s) present in the concentrate”, is meant the quantity of molecule(s) of alkyl polyglucoside(s) of formula (III), and more particularly (IV) present in the concentrate according to the invention.

Preferably, the total quantity of alkyl polyglucoside(s) is comprised between 0.5 and 15% by weight, more preferentially between 1 and 10% by weight, with respect to the total weight of the concentrate.

Advantageously, the total quantity of MEL(s) in the concentrate according to the invention is comprised between 0.1 and 25% by weight with respect to the total weight of the concentrate.

By “total quantity of MEL(s) present in the concentrate”, is meant the quantity of molecule(s) of MEL(s) of formula (I) or more particularly of formula (II) present in the concentrate according to the invention.

Preferably, the total quantity of MEL(s) is comprised between 0.5 and 10% by weight, more preferably between 1 and 5% by weight, with respect to the total weight of the concentrate.

Advantageously, the quantity of monopropylene glycol in the concentrate according to the invention is between 75 and 99.8% by weight with respect to the total weight of the concentrate.

In the concentrate according to the invention, the monopropylene glycol:MEL(s) ratio and/or the monopropylene glycol:alkyl polyglucoside(s) ratio is/are advantageously comprised between 0.5 and 1000.

The ratios indicated above refer to the total quantity of MEL(s) and to the total quantity of alkyl polyglucoside(s), respectively.

Preferably, each of the ratios monopropylene glycol:MEL(s) and/or monopropylene glycol:alkyl polyglucoside(s) is individually comprised between 1 and 500, more preferentially between 10 and 50.

As indicated above, there are several classes of MEL(s).

Advantageously, the concentrate according to the invention comprises at least two MELs selected from the group constituted by MEL-A, MEL-B, MEL-C and MEL-D.

Preferably, the concentrate according to the invention comprises MEL(s)-A, MEL(s)-B, MEL(s)-C and optionally MEL(s)-D, more preferentially MEL(s)-A, MEL(s)-B, MEL(s)-C and MEL(s)-D.

Advantageously, the concentrate according to the invention comprises MELs-A and MELs-B at a content comprised between 50 and 90% by weight, preferably comprised between 60 and 85% by weight, the percentages by weight being indicated with respect to the weight of the total quantity of MELs.

Advantageously, the concentrate according to the invention comprises MELs-C at a content greater than or equal to 5% by weight, preferably greater than or equal to 10% by weight, the percentages by weight being indicated with respect to the weight of the total quantity of MELs.

More particularly, the concentrate according to the invention can comprise MELs-A and MELs-B at a content comprised between 60% and 75% by weight, and MELs-C at a content greater than or equal to 10% by weight, preferably at a content greater than or equal to 20% by weight, the percentages by weight being indicated with respect to the weight of the total quantity of MELs.

Advantageously, the concentrate according to the invention also comprises at least one free fatty acid and/or at least one triglyceride.

By “free fatty acid”, is meant any fatty acid molecule that is not bound to another molecule. By “fatty acid” is meant any fatty acid molecule bound to another molecule, for example when this fatty acid molecule is present in a triglyceride or in a MEL.

The at least one free fatty acid and/or at least one triglyceride can have been introduced concomitantly with the at least one MEL.

In fact, depending on the process for obtaining the MELs, such as the fermentation process described above, and in particular depending on the separation method(s) implemented in the recovery step, the latter can comprise one or more free fatty acid(s) and/or triglyceride(s).

For example, the quantity of free fatty acid(s) and/or of triglyceride(s) present in the concentrate according to the invention can be comprised between 0.001 and 35% by weight, preferably between 0.01 and 20% by weight, with respect to the total weight of the concentrate.

More particularly, the concentrate comprises at least one free fatty acid and at least one triglyceride. In this case, the quantity of free fatty acid(s) and of triglyceride(s) present in the concentrate according to the invention can be comprised between 0.1 and 15% by weight, preferably between 0.2 and 10% by weight, more preferentially between 0.5 and 5% by weight, with respect to the total weight of the concentrate.

Advantageously, the free fatty acid(s) comprise(s) a carbon chain comprising between 14 and 24 carbon atoms, preferably 16 or 18 carbon atoms.

Advantageously, the triglyceride(s) comprise fatty acids comprising a carbon chain comprising between 14 and 24 carbon atoms, preferably 16 or 18 carbon atoms.

More particularly, in the present application, and in particular in the examples, when the MELs, at the end of the recovery step, comprise at least one free fatty acid, at least one triglyceride, water and/or strains, this mixture is called “mixture of MELs”.

In this case, the free fatty acid(s) and/or triglyceride(s) can originate from the residual oil present with the MEL(s) at the end of the fermentation process described above, said residual oil being the oil utilized as a carbon source in the fermentation process, which has not been used by the strains. In addition, the free fatty acid(s) can originate from the metabolism, by the strains, of the triglycerides comprised in the oil utilized as a carbon source in said process.

Moreover, according to the process for obtaining MELs, such as the fermentation process described above, and in particular according to the separation method(s) implemented in the recovery step, the MELs can also comprise water and fungal strains, more particularly yeast strains.

According to a preferred embodiment of the concentrate according to the invention, it comprises a mixture of MELs having the following characteristics:
a content of MELs greater than or equal to 40% by weight, preferably greater than or equal to 50% by weight, more preferentially greater than or equal to 55% by weight;
a content of other components less than or equal to 60% by weight, preferably less than or equal to 50% by weight, more preferentially less than or equal to 45% by weight (including free fatty acids, triglycerides, water and/or strains),
   the percentages by weight being given with respect to the total weight of the mixture of MELs.

Advantageously, in this preferred embodiment, the content of water and/or strains is less than 3% by weight, with respect to the total weight of the mixture of MELs.

This mixture of MELs can in particular be obtained by the fermentation process described above.

An example of a mixture of MELs and the process for obtaining it is also described in the following publication:
“Downstream processing of mannosylerythritol lipids produced by Pseudozyma aphidis”; Rau et al.; European Journal of Lipids Science and Technology 107 (2005) 373-380.

Preferably, a mixture of MELs comprises MELs of different classes, in general at least MELs-A, B and C. Preferentially, this mixture of MELs comprises MELs-A, B, C and D.

Moreover, a mixture of MELs advantageously comprises MELs-A and MELs-B at a content comprised between 50 and 90% by weight, preferably comprised between 60 and 85% by weight, the percentages by weight being indicated with respect to the weight of the total quantity of MELs.

In addition, a mixture of MELs advantageously comprises MELs-C at a content greater than or equal to 5% by weight, preferably greater than or equal to 10% by weight, the percentages by weight being indicated with respect to the weight of the total quantity of MELs.

More particularly, a mixture of MELs can comprise MELs-A and MELs-B at a content comprised between 60% and 75% by weight, and MELs-C at a content greater than or equal to 20% by weight, the percentages by weight being indicated with respect to the weight of the total quantity of MELs.

Such mixtures of MELs are for example obtained using a fermentation process such as those described above.

It is also possible to obtain a mixture of MELs having a content of MELs greater than or equal to 95%, preferably greater than or equal to 98% by weight, with respect to the total weight of the mixture of MELs. This mixture of MELs can, for example, be obtained using the fermentation process described above to which a purification step is added, at the end of the recovery step. This purification step can comprise a liquid/liquid extraction and/or a passing over a mineral substrate. Passing over a mineral substrate can be a chromatography, such as an adsorption chromatography on a silica column, carried out using suitable solvents. Such solvents are known to a person skilled in the art.

According to a preferred alternative embodiment of the concentrate according to the invention, it can therefore also comprise a mixture of MELs which has the following characteristics:
a content of MELs greater than or equal to 95% by weight, preferably greater than or equal to 98% by weight,
   the percentages by weight being given with respect to the total weight of the mixture of MELs.

Moreover, the purification step, following the recovery step of the MELs, can be carried out so as to obtain one class of MELs or even one MEL, at a content greater than or equal to 50%. By way of example, this purification step can comprise a liquid/liquid extraction and/or passing over a mineral substrate (such as a chromatography), as defined above.

The invention further relates to a process for the preparation of a concentrate according to the invention, comprising a step of mixing at least one MEL with at least one alkyl polyglucoside and monopropylene glycol.

The concentrates according to the invention can be prepared easily, by simple mixing of the components.

Advantageously, the mixing is carried out at ambient temperature under normal temperature and pressure (NTP) conditions.

Preferably, during the mixing, the components are heated at a temperature comprised between 25 and 55° C., more preferentially between 30 and 50° C., even more preferentially between 35 and 45° C.

Heating the components can allow a better homogenization of the concentrate according to the invention.

Optionally, prior to mixing, the process for the preparation of the concentrate according to the invention comprises obtaining at least one MEL, such as it is described above.

Advantageously, the MEL(s) are as described above and can be obtained by the fermentation process of MEL(s) described above, optionally followed by a purification step.

Advantageously, the alkyl polyglucoside(s) and the monopropylene glycol used in the process have the characteristics of these components as described above.

The invention also relates to a phytosanitary composition comprising a concentrate according to the invention, and a pesticide active ingredient.

Advantageously, the pesticide active ingredient is selected from the following active ingredients: herbicides, fungicides, insecticides, acaricides, growth regulators, insect repellents, and/or plant defense stimulators.

Preferably, the pesticide active ingredient is a herbicide active ingredient, a fungicide active ingredient, an insecticide active ingredient and/or a plant defense stimulator active ingredient.

Advantageously, the phytosanitary composition according to the invention comprises:
one or more fungicide active ingredients such as a carboxamide, a strobilurin, an azole (triazole, imidazole), a heterocyclic compound (pyridine, pyrimidine, piperazine, morpholine), a carbamate, an essential oil (cinnamaldehyde, thymol, tea oil), a microorganism (fungi, yeasts, bacteria) other than those capable of being comprised in the mixture of MELs described above, a polysaccharide (chitosan) and/or,
one or more herbicide active ingredients such as a lipid biosynthesis inhibitor, an acetolactase synthase inhibitor (also called “ALS inhibitor”), a photosynthesis inhibitor, an acetamide, a derivative of amino acids, an aryloxyphenoxypropionate, bipyridyl, cyclohexanedione, a dinitroaniline, diphenyl ether, hydroxybenzonitrile, imidazolinone, a phenoxyacetic acid, pyrazine, pyridine, a sulphonylurea, a triazine, a urea, a carbamate, a fatty acid of natural origin (caprylic acid, pelargonic acid) or derivatives thereof (salts, soaps) having herbicidal activity, and/or
one or more insecticide active ingredients such as an organo(thio)phosphate, a carbamate, a pyrethroid, a growth regulator of insects, an agonist/antagonist of the nicotinic receptors, an antagonist of GABA, a macrocyclic lactone, geraniol, eugenol, neem oil.

Advantageously, the pesticide active ingredient is of natural origin. Such pesticide active ingredients are generally called biopesticide active ingredients or biocontrol active ingredients.

Moreover, it will be noted that the active ingredient comprised in the phytosanitary composition according to the invention can have at the same time several of the following properties: herbicide, fungicide, insecticide, acaricide, growth regulator, insect repellent and/or plant defense stimulator.

Advantageously, the quantity of each pesticide active ingredient is comprised between 0.1 and 30% by weight, preferably between 1 and 20% by weight, more preferentially between 5 and 15% by weight with respect to the total weight of the phytosanitary composition.

The invention further relates to a cosmetic composition comprising a concentrate according to the invention, and a cosmetic active ingredient.

Advantageously, the cosmetic solution according to the invention comprises a cosmetic active ingredient selected from:
a moisturizer such as glycerin, fructose, butylene glycol and/or,
an organic UV filter such as PABA, PARA, a salicylate, a cinnamate, an anthranilate, benzophenone-3, butyl methoxydibenzoylethane, ethylhexyl triazone, drometrizole trisiloxane, diethylhexyl butamido triazone, 4-methylbenzylidene camphor, bemotrizinol, diethylamine hydroxybenzoyl hexyl benzoate, phenyl salicylate, methylene bis-benzotriazolyl tetramethylbutylphenol, benzophenone-1, benzophenone-2, benzophenone-8, bis-ethylhexyloxyphenol methoxyphenyl triazine, or a mineral UV filter and/or,
an anti-ageing agent such as a retinoid, an α- and β-hydroxy acid, a water-soluble vitamin, ascorbyl palmitate, a ceramide, a pseudo ceramide, a phospholipid, cholesterol, a sterol and/or,
an anti-cellulite agent such as isobutylmethylxanthine, theophylline, and/or
an anti-acne agent such as resorcinol, resorcinol acetate, benzoyl peroxide, salicylic acid, azelaic acid, and/or
a firming agent such as a plant extract (linseed extract), rose water, and/or
a vitamin such as vitamin A, derivatives thereof, vitamin B2, pantothenic acid, vitamin D, vitamin E.

Advantageously, the quantity of cosmetic active ingredient is comprised between 0.1 and 30% by weight, preferably between 1 and 20% by weight, even more preferentially between 5 and 15% by weight with respect to the total weight of the cosmetic composition.

The invention also relates to a process for the preparation of a phytosanitary composition according to the invention or a cosmetic composition according to the invention, comprising a step of mixing a concentrate according to the invention with an active ingredient.

More Particularly:

In the context of the preparation of a phytosanitary composition, the process according to the invention comprises a step of mixing a concentrate according to the invention with a pesticide active ingredient;

In the context of the preparation of a cosmetic composition, the process according to the invention comprises a step of mixing a concentrate according to the invention with a cosmetic active ingredient.

Advantageously, the components used in this process have the preferred characteristics of these components as described above.

The invention also relates to a solution comprising a concentrate according to the invention, a phytosanitary composition according to the invention, or a cosmetic composition according to the invention, and water.

As indicated above, by “solution” is meant in particular an aqueous solution, homogeneous or non-homogeneous. As examples of non-homogeneous aqueous solution, an emulsion, a dispersion or a suspension may be mentioned.

Advantageously, the solution according to the invention is a dispersion.

When the solution is prepared from a concentrate according to the invention, the water is selected depending on the use envisaged for the solution.

When the solution is prepared from a composition according to the invention, the water is chosen depending on the nature of said composition.

For example, in the context of the preparation of a solution comprising a phytosanitary composition according to the invention, the water is of the type used in the preparation of phytosanitary solutions, such as a drill water, which can be a water having a hardness from medium to hard. Advantageously, the water having a hardness from medium to hard has a hardness comprised between 300 and 600 ppm, preferentially between 450 and 550 ppm. Such a solution is generally intended to be sprayed, for example by a farmer on crops.

In the context of the preparation of a solution comprising a cosmetic composition according to the invention, the water is a water generally used in cosmetics, such as a distilled water or a water treated by reverse osmosis.

The invention also relates to a process for the preparation of a solution according to the invention, comprising a step of mixing a concentrate according to the invention, a phytosanitary composition according to the invention, or a cosmetic composition according to the invention, with water.

The solutions according to the invention may also be prepared easily by simple mixing of the concentrates or of the compositions according to the invention with water.

Advantageously, the volume ratio of concentrate or of composition according to the invention to water is between 1:400 and 1:20, preferably between 1:300 and 1:30, even more preferentially between 1:250 and 1:40.

The invention also relates to the use of a concentrate according to the invention as adjuvant. As stated above, by “adjuvant” is meant any substance that has the role of improving the properties of a solution, such as a phytosanitary solution.

More particularly, the concentrate according to the invention is used as a wetting agent.

The concentrate according to the invention can be used as a wetting agent in the preparation of any type of solutions in which it is usual to use wetting agents. As an example, the concentrate according to the invention can be used in cleaning solutions, such as solutions for household maintenance.

Advantageously, the concentrate according to the invention is used as a wetting agent in a phytosanitary solution, such as a pesticide solution.

Furthermore, the concentrate according to the invention can be used as a wetting agent in a cosmetic solution.

Moreover, the concentrate according to the invention can be used in various other applications, such as in fire pumps.

The invention will be better understood in light of the examples which follow, given by way of illustration, with reference to the following figures:

FIG. 1, which represents the contact angle Θ formed by the tangent to a drop of liquid 1 at the point of contact with a flat solid surface 2;

FIG. 2, which represents three cases in point showing the idea of wetting, namely the case of a total wetting of a flat solid surface 2 by a drop of liquid 1 (FIG. 2a), the case of a partial wetting of a flat solid surface 2 by a drop of liquid 1 (FIG. 2b) and the case where a drop of liquid 1 does not wet a flat solid surface 2 (FIG. 2c);

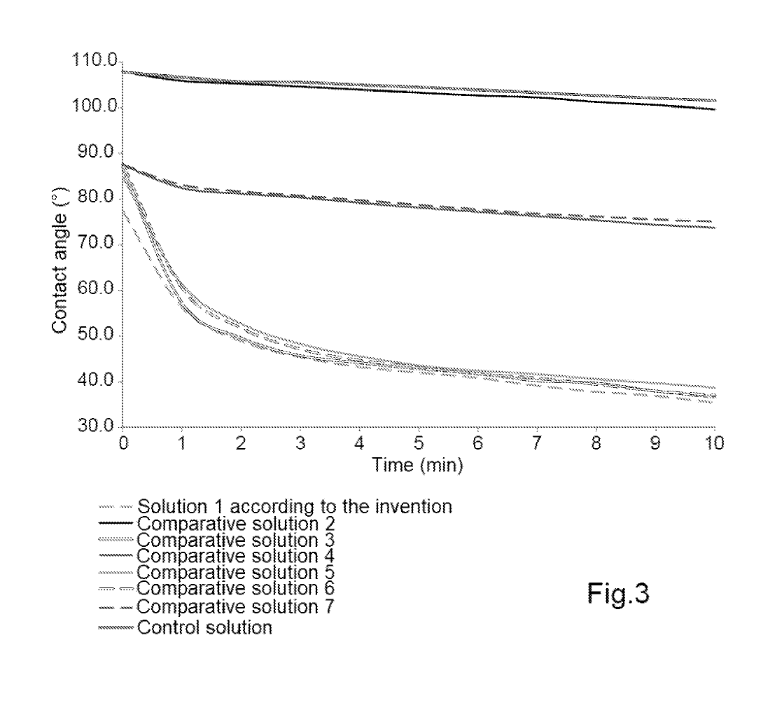

FIG. 3, which is a diagram representing the reduction in the contact angle obtained with a solution comprising the concentrate according to the invention and solutions comprising comparative concentrates.

### EXAMPLE 1; PREPARATION OF A CONCENTRATE ACCORDING TO THE INVENTION

1. Obtaining the MELs

The MELs were obtained by a fermentation process comprising the following steps:
culturing a yeast strain such as Pseudozyma aphidis in the presence of vegetable oil (rapeseed) in order to obtain the MELs; and
recovering the MELs thus obtained.

At the end of the step of recovering the MELs, a mixture of MELs is obtained which has the following characteristics:
Content of MELs: 55% by weight
Content of other components: 45% by weight (of which 42% by weight of free fatty acids and triglycerides and 3% by weight of water and strain),
   the percentages by weight being given with respect to the total weight of the mixture of MELs obtained.

2. Alkyl Polyglucosides

Simulsol® SL11W from Seppic was used. Simulsol® SL11W is mainly composed (approximately 40 to 60% by weight) of alkyl polyglucosides of the following formula (IV):

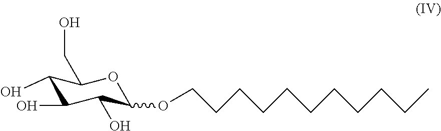

3. Monopropylene Glycol

Monopropylene glycol from OLEON marketed under the trademark RADIA® 4713 was used.

4. Process for the Preparation of the Concentrate According to the Invention

5% by weight of the mixture of MELs, 5% by weight of Simulsol® S11W and 90% by weight of monopropylene glycol were added to a 60-mL glass flask, the percentages by weight being indicated with respect to the total weight of the concentrate obtained, and then stirred manually until homogenization of the concentrate was achieved. During stirring, it is possible to heat the concentrate at 40° C. in order to facilitate the homogenization.

### EXAMPLE 2 EVALUATION OF THE WETTING POWER OF A SOLUTION COMPRISING THE CONCENTRATE ACCORDING TO THE INVENTION AND OF SOLUTIONS COMPRISING COMPARATIVE CONCENTRATES—SYNERGISTIC EFFECT

In order to evaluate the effect of the concentrate according to the invention on the wetting power of a solution, an aqueous solution comprising the concentrate according to the invention and aqueous solutions comprising comparative concentrates were prepared. Measurements of the contact angle of the aqueous solutions obtained were then carried out.

1. Materials and Methods

1.1 Materials

The following products were used in this Example:
monopropylene glycol (RADIANOL® 4713, OLEON)
alkyl polyglucosides (Simulsol® SL11W)
the mixture of MELs prepared in Example 1
the concentrate according to the invention prepared in Example 1
hard water (hardness equal to 500 ppm)

The following equipment was also used in this Example:
glass bottles,
1-mL syringes equipped with a needle with a diameter of 0.63 mm
hydrophobic parafilms (Parafilm “M”, NEENAH, Wis. 54956)
the DSA10 goniometer (KRUSS)
the “Drop shape analysis” software (KRUSS)

1.2. Methods

Preparation of the Aqueous Solution Comprising the Concentrate According to the Invention

0.500% by weight of the concentrate according to the invention was added to 99.500% by weight of water in a glass flask, the percentages by weight being indicated with respect to the total weight of the solution obtained, and then stirred manually until homogenization of the solution was achieved.

Preparation of the Aqueous Solutions Comprising Comparative Concentrates

Comparative Solution 2

0.450% by weight of monopropylene glycol was added to 99.550% by weight of water in a glass flask, the percentages by weight being indicated with respect to the total weight of the solution obtained, and then stirred manually until homogenization of the solution was achieved.

Comparative Solution 3

0.025% by weight of the mixture of MELs 1 was added to 99.975% by weight of water in a glass flask, the percentages by weight being indicated with respect to the total weight of the solution obtained, and then stirred manually until homogenization of the solution was achieved.

Comparative Solution 4

0.025% by weight of Simulsol® SL11W was added to 99.975% by weight of water in a glass flask, the percentages by weight being indicated with respect to the total weight of the solution obtained, and then stirred manually until homogenization of the solution was achieved.

Comparative Solution 5

0.025% by weight of Simulsol® SL11W and 0.025% by weight of the mixture of MELs 1 were added to 99.950% by weight of water in a glass flask, the percentages by weight being indicated with respect to the total weight of the solution obtained, and then stirred manually until homogenization of the solution was achieved.

Comparative Solution 6

0.025% by weight of the mixture of MELs 1 and 0.475% by weight of MPG were added to 99.5% by weight of water in a glass flask, the percentages by weight being indicated with respect to the total weight of the solution obtained, and then stirred manually until homogenization of the solution.

Comparative Solution 7

0.025% by weight of Simulsol® SL11W and 0.475% by weight of monopropylene glycol were added to 99.5% by weight of water in a glass flask, the percentages by weight being indicated with respect to the total weight of the solution obtained, and then stirred manually until homogenization of the solution was achieved.

The various solutions prepared are summarized below in Table 1:

TABLE 1
Solutions prepared in Example 2.

| | |
|---|---|
| Solution 1 | Concentrate according to the invention + water |
| Comparative solution 2 | Monopropylene glycol + water |
| Comparative solution 3 | Mixture of MELs + water |
| Comparative solution 4 | Simulsol ® SL11W + water |
| Comparative solution 5 | Simulsol ® SL11W + Mixture of MELs 1 + |
| | water |
| Comparative solution 6 | Mixture of MELs + Monopropylene glycol + |
| | water |
| Comparative solution 7 | Simulsol ® SL11W + Monopropylene glycol + |
| | water |

A control solution only comprising water was also prepared.

Measurement of the Contact Angles

Contact angle measurements were carried out for each of solutions 1 to 7 and the control solution, using the goniometer.

To this end, a drop of each emulsion and of the control solution (3 μL) was formed using the syringe. The syringe was then placed approximately 0.5 cm above the hydrophobic parafilm. By gravity, this drop became detached from the needle and fell onto the hydrophobic parafilm. The variation in the contact angle was monitored for 10 minutes from the moment when the drop touched the parafilm, using the analysis software.

The results were then processed in order to compare the variations in contact angles for each of solutions 1 to 7, with respect to the control solution.

2. Results

The results of the contact angle measurements for each of solutions 1 to 7 and for the control solution are presented in FIG. 3.

The results show that the reduction in the contact angle obtained with solution 1 is greater than that obtained with each of solutions 2 to 7. Solution 1, comprising the concentrate according to the invention, therefore has greater wetting power than solutions 2 to 7 comprising comparative concentrates.

Thus, a solution comprising the concentrate according to the invention, i.e. comprising a combination of at least one alkyl polyglucoside, at least one MEL and monopropylene glycol, has greater wetting power than a solution comprising one, or a combination of two of these three components.

In particular, the addition of monopropylene glycol to the mixture of MELs (comparative solution 6), or the addition of monopropylene glycol to Simulsol® SL11W (comparative solution 7) does not have any positive effect on the reduction in contact angle, with respect to the reductions in contact angle obtained with the mixture of MELs alone (comparative solution 3) or Simulsol SL11W alone (comparative solution 4).

Now, surprisingly, the combination of monopropylene glycol, mixture of MELs and Simulsol® SL11W (solution 1) results in a reduction in the contact angle greater than that obtained with the mixture of MELs alone (comparative solution 3), Simulsol® SL11W alone (comparative solution 4), and more particularly with the mixture of MELs combined with Simulsol® SL11W (comparative solution 5).

Thus, surprisingly, when monopropylene glycol is added to a solution comprising at least one alkyl polyglucoside and at least one MEL, so that the solution comprises the concentrate according to the invention, the wetting power of said solution is increased considerably.

Surprisingly, the results presented in FIG. 3 show that the reduction in the contact angle obtained with solution 1 comprising the concentrate according to the invention (monopropylene glycol, mixture of MELs, Simulsol® SL11W) is greater than the sum of the reductions obtained with comparative solution 2 (comprising monopropylene glycol) and comparative solution 5 (comprising the mixture of MELs and Simulsol® SL11W). This synergistic effect is particularly evident at the moment when the drop is deposited on the hydrophobic film (T0), as shown in Table 2 below.

TABLE 2
Synergy observed between the components of the
concentrate according to the invention at T0

| | Control | Solution | Solution | Solution |
|---|---|---|---|---|
| | solution | 2 | 5 | 1 |
| Contact angle at T0 (°) | 107.8 | 107.9 | 84.9 | 77.5 |

Thus, when a drop of a solution comprising the concentrate according to the invention is deposited on a hydrophobic surface, such as a leaf, it spreads more effectively over the leaf at the moment of impact and is therefore less likely to bounce and land outside of said leaf. This is particularly advantageous when spraying phytosanitary solutions.

### EXAMPLE 3: USE OF CONCENTRATE ACCORDING TO THE INVENTION IN THE PREPARATION OF A PHYTOSANITARY COMPOSITION—PREPARATION OF A SOLUTION

A concentrate according to the invention was prepared, then used in the preparation of a phytosanitary composition.

The concentrate according to the invention has the following characteristics:

| Components | % by weight* |
|---|---|
| Alkyl polyglucosides | 6 |
| Mixture of MELs | 8 |
| Monopropylene glycol | 86 |

*Percentage by weight with respect to the total weight of the concentrate

A phytosanitary composition was then prepared, by mixing the concentrate according to the invention with cinnamaldehyde. The phytosanitary composition according to the invention obtained has the following characteristics:

| Components | % by weight* |
|---|---|
| Concentrate according to the invention | 90 |
| Cinnamaldehyde (Herbarom Laboratory) | 10 |

*Percentage by weight with respect to the total weight of the phytosanitary composition

A solution was then prepared from the phytosanitary composition according to the invention obtained, as follows:

50% by weight of water, 1% by weight of the phytosanitary composition according to the invention and then 49% by weight of water were successively added into a resealable container, the percentages by weight being indicated with respect to the total weight of the solution obtained. If necessary, the pH and the salinity of the water will have been adjusted beforehand.

The solution according to the invention obtained has the following characteristics:

| Components | % by weight* |
|---|---|
| Phytosanitary composition | 1 |
| according to the invention | |
| water | 99 |

*Percentage by weight with respect to the total weight of the solution

### EXAMPLE 4: USE OF A CONCENTRATE ACCORDING TO THE INVENTION IN THE PREPARATION OF A COSMETIC COMPOSITION PREPARATION OF A SOLUTION

A concentrate according to the invention was prepared, then used in the preparation of a cosmetic composition.

The concentrate according to the invention has the following characteristics:

| Components | % by weight* |
|---|---|
| Alkyl polyglucosides | 8 |
| Mixture of MELs | 8 |
| Monopropylene glycol | 84 |

*Percentage by weight with respect to the total weight of the concentrate

A cosmetic composition was then prepared by mixing the concentrate according to the invention with a linseed extract. The cosmetic composition according to the invention obtained is a firming composition for the face, which has the following characteristics:

| Components | % by weight* |
|---|---|
| Concentrate according to the invention | 90 |
| Linseed extract (Aroma-zone) | 10 |

*Percentage by weight with respect to the total weight of the cosmetic composition

A solution was then prepared from the cosmetic composition according to the invention obtained, as follows:

5% by weight of the cosmetic composition according to the invention was added to 95% by weight of water in a flask, the percentages by weight being indicated with respect to the total weight of the cosmetic solution obtained. If necessary, the pH and the salinity of the water will have been adjusted beforehand.

The firming solution for the face according to the invention obtained has the following characteristics:

| Components | % by weight* |
|---|---|
| Cosmetic composition | 5 |
| according to the invention | |
| Water | 95 |

*Percentage by weight with respect to the total weight of the solution

Other components may be added to this solution, such as other cosmetic active ingredients and/or formulation agents.

1. A concentrate comprising:
at least one mannosylerythritol lipid (MEL);
at least one alkyl polyglucoside, and
monopropylene glycol.

2. The concentrate according to claim 1, in which the at least one alkyl polyglucoside is a molecule of the following formula (III): 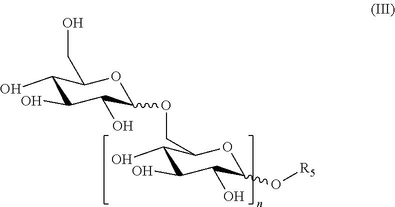
in which:
n is an integer comprised between 0 and 5,
R₅ is an alkyl chain comprising between 5 and 20 carbon atoms.

3. The concentrate according to claim 1, in which the total quantity of alkyl polyglucoside(s) is comprised between 0.1 and 25% by weight with respect to the total weight of the concentrate.

4. The concentrate according to claim 1, in which the total quantity of MEL(s) is comprised between 0.1 and 25% by weight with respect to the total weight of the concentrate.

5. The concentrate according to claim 1, in which the quantity of monopropylene glycol is comprised between 75 and 99.8% by weight with respect to the total weight of the concentrate.

6. The concentrate according to claim 1, in which the monopropylene glycol:MEL(s) ratio and/or the monopropylene glycol:alkyl polyglucoside(s) ratio is/are comprised between 0.5 and 1000.

7. The concentrate according to claim 1, comprising at least two MELs selected from the group consisting of MEL-A, MEL-B, MEL-C and MEL-D.

8. The concentrate according to claim 1, further comprising at least one free fatty acid and/or at least one triglyceride.

9. A process for the preparation of a concentrate according to claim 1, comprising mixing at least one MEL with at least one alkyl polyglucoside and monopropylene glycol.

10. A phytosanitary composition comprising a concentrate according to claim 1, and a pesticide active ingredient.

11. A cosmetic composition comprising a concentrate according to claim 1, and a cosmetic active ingredient.

12. A solution comprising a concentrate according to claim 1 and water.

13. A process for the preparation of a solution claim 12, comprising mixing a concentrate according to claim 1 with water.

14. (canceled)

15. A solution comprising a phytosanitary composition according to claim 10 and water.

16. A solution comprising a cosmetic composition according to claim 11 and water.

17. A process for the preparation of a solution, comprising mixing a phytosanitary composition according to claim 10 with water.

18. A process for the preparation of a solution, comprising mixing a cosmetic composition according to claim 11 with water.
